(19) United States
(12) Patent Application Publication
(10) Pub. No.: US 2010/0170505 A1
(43) Pub. Date: Jul. 8, 2010

(54) DEVICE FOR ENHANCING WELL-BEING

(76) Inventor: Bryan Barker SPEIGHT, Durban (ZA)

Correspondence Address: YOUNG & THOMPSON, 209 Madison Street, Suite 500, Alexandria, VA, 22314, US

(21) Appl. No.: 12/686,555

(22) Filed: Jan. 13, 2010

Related U.S. Application Data
(63) Continuation-in-part of application No. 10/535,149, filed on Jan. 6, 2006, now abandoned.

(30) Foreign Application Priority Data
Nov. 15, 2002 (ZA) 2002/3851

(51) Int. Cl. A61M 16/00

(52) U.S. Cl. 128/200.24

(57) ABSTRACT

A method of creating well being using an inhaling device. The inhaling device is provided which has an inlet for oxygen or air containing oxygen, and an outlet in the form of a mouthpiece. A permanent magnet of strength between about 1500 and 3000 gauss is located between the inlet and the mouthpiece so that the user can draw oxygen through the device into the mouth past the magnet which induces paramagnetism to the oxygen.

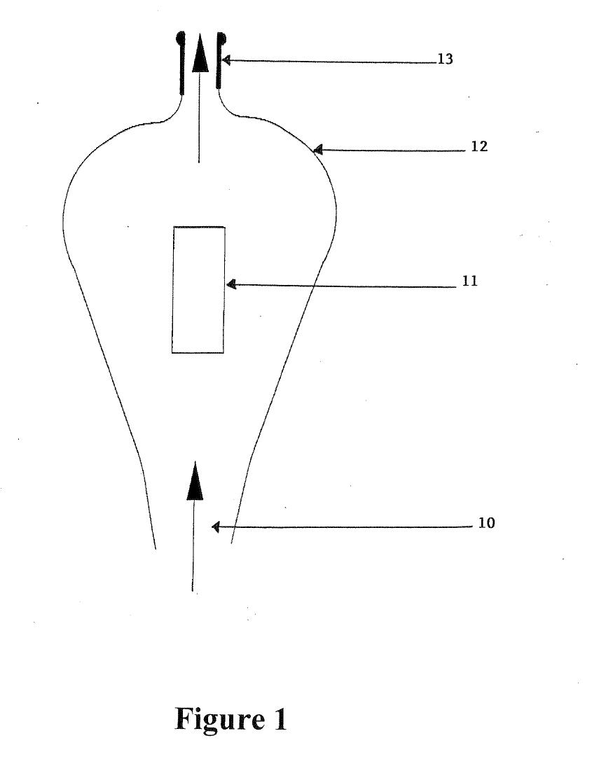

This is a continuation-in-part of U.S. patent application Ser. No. 10/535,149, filed Jan. 9, 2006, which is the 371 National Stage application of International application no. PCT/ZA2003/000170, filed Nov. 12, 2003, which claims priority to South African application no. 2002/3851, filed Nov. 15, 2002. The entire contents of the above-referenced applications are hereby incorporated by reference in their entirety.

### TECHNICAL FIELD OF THE INVENTION

This invention relates to a method for enhancing the well-being of humans.

The term “well-being” is chosen to include the alleviation of disease and other physiological problems, as well as to improve performance in many aspects of life such as sport and other functions; and also to contribute to the regulation of the immune system.

In a particular application of the invention the treatment of asthma and emphasemia has been examined.

### BACKGROUND ART

Research has been carried out on the absorption or adsorption of oxygen to the iron sites of the haemoglobin molecule. Thus, oxygen molecules cross the alveolar-capillary membrane and are dissolved in the plasma. The amount of dissolved oxygen in the plasma is known to be important and it is the haemoglobin that is responsible for the amount of oxygen in the blood. Approximately 1.3 ml of oxygen dissolve in 1 gm of haemoglobin.

It is an object of the present invention to provide a method which maximises the entry of oxygen into the plasma and attachment or oxygen onto the haemoglobin cells to form oxy-haemoglobin.

### SUMMARY OF THE INVENTION

According to the invention a method of creating well-being in a human is provided which causes air to be drawn past a permanent magnet having a magnetic field of about 1500 to 3000 gauss located in a passage having an inlet for air and an outlet for air containing paramagnetic oxygen; and causing the paramagnetic oxygen to enter the respiratory organs of the human.

The invention is of particular application in the treatment of a human suffering from asthma, of increasing athletic performance and stimulating immunity.

### BRIEF DESCRIPTION OF THE DRAWING

An example of the invention is illustrated in the accompanying drawing which is cross-section of a device for use in the method according to the invention. The device includes an inlet (10) for air, a body (12) including a permanent magnet (11) of strength above 1500 gauss and a mouthpiece (13). With the mouthpiece in position, the patient draws air through the body and past the magnet, thereby inducing paramagnetism in the oxygen.

### DETAILED DESCRIPTION OF THE INVENTION

In a preferred form of the invention the magnetic field is created by a permanent magnet, electromagnet or other source of magnetic field in the device, and the strength of the magnetic field is preferably but not limited to the order of 1,500 gauss to 3,000 gauss.

The arrangement of inlet and mouthpiece is designed for the person using the device to draw air through the device but it will be appreciated that means may be provided to assist the passage of the oxygen-containing gas through the device. This may be particularly useful in anaesthesiology by providing the patient with increased oxygen supply during anaesthetic procedures.

Experiments have shown that use of the device leads to a definite improvement to the immune system and there have also been exciting improvements in the enhancement of performance and well-being.

A number of surveys were conducted to support the effectivity of the invention.

### Survey 1

Fourteen athletes were selected for the survey. Ten were supplied with a device (called THERAHALER, (i.e. an inhaler device)) according to the invention and four were not. The results are given in the following table.

TABLE

| | ATHLETES USING | | | | | |
|---|---|---|---|---|---|---|
| | THERAHALER | | | ATHLETES USING PLACEBO | | |
| | (10) | | | (4) | | |
| | AT | AFTER 4 | | AT | AFTER 4 | |
| TEST PARAMETER | START | WEEKS | CHANGE | START | WEEKS | CHANGE |
| RESTING HEART RATE | 66.14 | 61.8 | −7.35% | 64.75 | 66.5 | +1.02% |
| (BEATS PER MINUTE | | | | | | |
| AVERAGE) | | | | | | |
| HEART RATE AFTER 15 | 160.2 | 150.5 | −6.0% | 163.75 | 165.00 | −0.91% |
| MINUTES EXERCISE | | | | | | |
| ROUTINE (BEATS PER | | | | | | |
| MINUTE AVERAGE | | | | | | |
| ATHLETE) | | | | | | |
| AFTER 1 MINUTE REST | 122.7 | 105.6 | −13.9% | 123.00 | 124.25 | +1.0% |
| (BEATS PER MINUTE) | | | | | | |
| AVERAGE PER ATHLETE | | | | | | |
| AFTER 3 MINUTES REST | 91.3 | 81.1 | −11.7% | 92.25 | 98.25 | +6.5% |
| (BEATS PER MINUTE) | | | | | | |
| AVERAGE PER ATHLETE | | | | | | |
| | | | | BREATH HOLDING TIME | | |
| BREATH HOLDING TIME (SECONDS) | | | | (SECONDS) | | |
| AVERAGE PER ATHLETE | 67.3 | 73.2 | +26.9 | 54.5 | 57.25 | +5% |

### Comments:

1. Test subjects: 14 above average athletes, volunteers from various athletic disciplines. 10 used THERAHALER every 30 minutes for 4 weeks, 4 used placebos.

2. Test subject's fitness/endurance capabilities were tested
a) at commencement
b) after 4 weeks using an exercise bicycle with variable, measurable loading as per chart and heart rate per minute was measured at intervals indicated. All subjects continued with normal training regime during the test period. (4 weeks)

3. The athletes were required to use their device thus:
a) Expel air from lungs.
b) Inhale atmospheric air through the THERAHALER/PLACEBO until lungs were full.
c) Hold breath as long as possible (measured in seconds)
d) Exhale
e) Repeat every 30 minutes during waking hours

### Survey 2

This survey was conducted on 28 top class rugby players—20 without THERAHALER and 8 using THERAHALER every 30 minutes.

The test used was the 20 m “Bleep Test” where a player is required to run 20 m between beacons, each lap a little faster than the last. When a participant cannot keep up the pace set by a bleep, he is disqualified.

The 28 players performed a total of 2643 laps (average 104-26 laps per player).

### Report ONE

Three weeks synopsis of 12 Players:

| Without | |
|---|---|
| THERAHLER (7 Players) | With THERAHALERS (5 Players) |
| Extra number of laps | Extra number of laps |
| completed . . .100 | completed . . .128 |
| Average Extra per | Average Extra per Player . . . 25.6 |
| Player . . . 14.29 | |
| Improvement as % of base | Improvement as % of base . . . 24.44% |
| (104.26) . . . 13.7% | |

### Report TWO

Synopsis of Performance Improvement of all 24 Players over 1 to 4 weeks:

| Without THERAHLER (16 Players) | With THERAHALERS (8 Players) |
|---|---|
| Total 38 Weeks Usage | Total 22 Weeks Usage |
| 255 Extra laps | 196 Extra Laps |
| Improved Laps per week . . . 6.7 | Improved Laps per week . . . 8.9 |

(2.2 Extra over Non THERAHALER Players)=2% Improvement in Performance

### Observations:

1. Players who use THERAHALER can expect to attain an extra 25% improvement in fitness levels after three weeks over player who do not use THERAHALER.

2. The greater percentage of THERAHALER players completing the three week course, held during a flu epidemic, would substantiate improve immune system function observed with the ASTHMATIC patient trial.

3. Players using THERAHALER reported an improved feeling of WELL BEING (as did ASTHMA patients) which indicates an improved confidence level and an improved all round state of health.

4. Tests using work load bicycle and measuring heart work & recovery rates yield supportive results, but in this rest, 25% placebos were used and they showed disappointing results—

### Survey 3

A quality of life study was completed by 45 asthmatic patients as required by protocol for Juniper Quality of Life Questionnaires (AQLQ).

The protocol was constructed as follows:
a. 14 day observation period to ascertain the stability of the patients condition.
b. 28 day intensive THERAHALER therapy (every 30 mins).
c. Second 28 day intensive THERAHALER Therapy.
d. 30 day maintenance THERAHALER Therapy (6× per day to establish whether the benefits gained in the 56 day intensive therapy period were lasting or not.
e. Final patient check up—it is at this period that these patient feedback reports are filled in.

The results of the response to the questions for the first and last visits of patients were analyzed to investigate whether there was any significant improvement in the quality of life as measured by the questions of the AQLQ(S) questionnaire.

A paired t-test was applied to each of the 32 questions. For most of the questions, the sample size was n=44 except in a few cases where a patient may not have answered a particular questions. All the questions, with the exception of question 12, showed a significant improvement. This is shown by the negative values of the t-statistic with accompanying p-values<0.01 for all questions but for question 4 which had a p-value=0.03<0.05 (The difference for the paired t-statistic was taken as d=score on visit 1—score on visit 5. A negative difference is an indication of an improvement).

Question 12 which asks “How much discomfort have you felt over the past 2 weeks as a result of coughing?” yielded t=−1.375 with a p-value of 0.176 and although not significant at a 5% level of significance, still indicates an improvement.

The statistical results thus show that the use of the THERAHALER has improved the patients' quality of life as measured by the AQLQ(S) questionnaire.

In Addition:

1 All patients report being able to breathe easier and can better perform their normal functions at work and home and enjoy an improved quality of life.

2 All patients had experienced frequent Asthma attacks—some near fatal before using THERAHALER since completing their eight week regimine.

3 With one exception, all patients have significantly reduced their medicine intake—two have stopped carrying their Bronchial dilator pumps around with them, and some have stopped using cortico steroids.

4 Three patients got flu and one bronchitis after completing the test and reported no deterioration in their asthma, indicating that their immune system was functioning normally.

5 Many patients can now enjoy foods, which they could not previously enjoy because it would trigger an asthma attack, again indicating that their immune systems have improved.

6 No adverse reactions or experiences were felt and all patients reported that they preferred using the THERAHALER because it is a non-medicated option utilizing natural principles.

7 Improvement in peak flow meter readings indicated an improvement in lung function.

8 Patients reported enjoying an uninterrupted nights sleep completing the THERAHALER regime, because wheezing and coughing had diminished or had ceased altogether.

### Survey 4

This survey was aimed to determine the effects of regular use of THERAHALER on arterial blood gas concentrations and T Cell numbers.

### Method

1. 7 THERAHALER board members were recruited for the study.

2. Blood sampling involved taking an arterial blood sample from the radial artery and a venous sample from the brachial vein for T Cell analysis.

3. The arterial blood sample was analysed for the following parameters:
i) Partial pressure of oxygen (PO2)
ii) Partial pressure of carbon dioxide (PCO2)
iii) Oxy-haemoglobin percentage (O2Hb)
iv) Carboxyhaemoglobin percentage (COHb)
v) Methaemaglobin percentage (metHb)
vi) Haemoglobin concentration (Hb)

4. The venous blood sample was analysed and the following counts were conducted:
i) CD3 Count
ii) CD4 Count
iii) CD8 Count

5. Baseline sampling (arterial and venous) was done on all subjects.

6. The test subjects were then instructed to use the THERAHALER every 5 minutes for the next two hours and repeat arterial sampling was conducted.

7. The subjects were then sent home and requested to use the THERAHALER as directed every 30 minutes while awake.

8. Further arterial and venous sampling was conducted.

### Pilot Blood Gas Investigation Results:

| | Commencement | | | |
|---|---|---|---|---|
| | (Before | After 2 hours | After 2 weeks | After 4 weeks |
| | THERAHALER) | (Continuous use) | (Every 30 minutes) | (Every 30 minutes) |
| Parameter | Average Reading | Average Reading | Average Reading | Average Reading |
| Oxy-haemoglobin | 93.69 | 94.00 | 94.30 | 94.75 |
| PO2 | 92.99 | 92.09 | 101.40 | 91.17 |
| PCO2 | 39.50 | 39.80 | 38.36 | 39.68 |
| Haemoglobin | 15.33 | 15.61 | 15.81 | 15.88 |

The object of the two tests was to investigate trends to gain a better understanding of how THERAHALER's magnetic field impacts blood physiology. The following findings were made:—

Oxy-haemoglobin: This showed a steady, incremental increase from a starting average of 93.69% to 94.75% four weeks later.

PO2 Levels: The test reveals a slight drop in PO2 levels over the four week period, but the third reading being rather erratic and should be ignored.

PCO2 Levels: Over the four week test, the levels remain almost constant, indicating that improvement in Oxy-haemoglobin levels are not as a result of hyperventilation.

Haemoglobin concentration G/dl: Here, surprisingly, small, but steady incremental increases in haemoglobin concentration from 15.33 to 15.83 were found. Normally, when oxy-haemoglobin levels increase haemoglobin levels decrease.

Conclusion: THERAHALER does improve blood oxygen levels without significantly disturbing blood CO2 levels.

### Survey 5: Test for Changes in Immunity Level Function Average Reading

Test Procedure: Seven trialists used the THERAHALER for a four week period and venous blood samples taken initially before THERAHALER usage, after two weeks, after four weeks. CD3, CD4, CD8 levels were noted at these intervals yielding the following results.

### Results

| | Commencement | | |
|---|---|---|---|
| | (Before | After 2 Weeks | After 4 Weeks |
| | THERHALER) | (Every 30 minutes) | (Every 30 minutes) |
| Parameter | Average Reading | Average Reading | Average Reading |
| CD3 | 1662.67 | 1373.5 | 1724 |
| CD4 | 982 | 784 | 1015 |
| CD8 | 634 | 906 | 675 |

Conclusion: CD3 and CD4 counts show a significant improvement whilst CD8 shows moderate improvement. When these results are correlated with patient reports from Survey 3 Asthma trials, many patients reported large reductions and in some cases cessation of corticosteroid drug therapy combined with an increase resistance to flu and bronchitis.

### Safety:

THERAHALER has no reported adverse effects during this test, or any previous tests, and has also proved to be completely compatible with all allotropic medicine regimes encountered to date. THERAHALER's safety and drug compatibility is one of the device's many outstanding features.

What is claimed is:

1. A method of creating well-being in a human comprising the step of causing air to be drawn past a permanent magnet having a magnetic field of about 1500 to 3000 gauss, to form a paramagnetic oxygen, and causing the paramagnetic oxygen to enter the respiratory organs of the human.

2. The method according to claim 1 wherein the well-being is selected from the group consisting of relief from asthma, stimulation of immunity and increase in athletic performance.
